(19) United States
(12) Patent Application Publication
(10) Pub. No.: US 2005/0124999 A1
(43) Pub. Date: Jun. 9, 2005

(54) Device and method for radial delivery of a structural element

(76) Inventors: George P. Teitelbaum, Santa Monica, CA (US); Donald W. Larsen, La Canada, CA (US)

Correspondence Address: HOGAN & HARTSON L.L.P., 500 S. GRAND AVENUE, SUITE 1900, LOS ANGELES, CA, 90071-2611, US

(21) Appl. No.: 10/979,405

(22) Filed: Nov. 1, 2004

Related U.S. Application Data
(60) Provisional application No. 60/516,326, filed on Oct. 31, 2003.

(51) Int. Cl. A61B 17/58

(52) U.S. Cl. 606/99

(57) ABSTRACT

A device for delivery of a structural element to a destination is disclosed. The device comprises a channel and a plunger slidably positioned inside the channel. The channel has a proximal end, a distal end, and a barrier formed across the channel at the distal end. The plunger is adapted to move the structural element axially through the channel from the proximal end to the distal end and to push it radially into the destination. Methods of percutaneous fixation of a spinal compression fracture and kits for use with the methods of the present invention are also disclosed.

This application claims priority to the U.S. Provisional Patent Application No. 60/516,326, filed on Oct. 31, 2003.

### FIELD OF THE INVENTION

This invention relates generally to the delivery of structural elements into a desired location and, in particular, to a device and methods of percutaneous fixation of a spinal compression fracture and reexpanding a partially collapsed vertebral body by seating a rod-like structure within the vertebral body.

### BACKGROUND OF THE INVENTION

Osteoporotic spinal compression fractures (crushing injuries to one or more vertebrae) represent a major health problem worldwide with as many as 700,000 injuries occurring annually in the United States. Until recently, the treatment of vertebral compression fractures has consisted of conservative measures including rest, analgesics, dietary, and medical regimens to restore bone density or prevent further bone loss, avoidance of injury, and bracing. Unfortunately, the typical patient is an elderly person who generally does not tolerate extended bed rest well. As a result, minimally invasive surgical methods for treating vertebral compression fractures have recently been introduced and are gaining popularity (U.S. Pat. No. 6,595,998).

One technique used to treat vertebral compression fractures is the injection of bone filler, such as polymethyl methacrylate (PMMA), into the fractured vertebral body. This procedure is commonly referred to as percutaneous vertebroplasty. But this procedure cannot be used to reestablish lost spinal column height.

Kyphoplasty is another vertebral fracture treatment that uses one or two balloons, similar to angioplasty balloons, to attempt to reduce the fracture and restore vertebral height prior to injecting the bone filler. Two balloons are typically introduced into the vertebra via bilateral transpedicular cannulae. The balloons are inflated to reduce the fracture. After the balloon(s) is deflated and removed, leaving a relatively empty cavity, bone cement is injected into the vertebra. In theory, the inflation of the balloons restores vertebral height. However, it is difficult to consistently attain meaningful height restoration. It appears the inconsistent results are due, in part, to the manner in which the balloon expands in a compressible media and the structural orientation of the trabecular bone within the vertebra (U.S. Pat. No. 6,595,998).

Recently, another approach to the treatment of the spinal compression fractures have been described in U.S. Pat. No. 6,595,998. The method involves consecutive inserting a plurality of wafers between the tissue surfaces to create a column of wafers. The column expands in a given direction as wafers are consecutively added to the column. However, this method appear to require application of axial force to move a pre-assembled column of the wafers into the bone, which may lead to an inadvertent perforation of the anterior cortex of the target vertebral body by the column. Moreover, the method requires a high precision in aligning and fitting the wafers on top of each other and, thus, is quite laborious.

There are many other physical conditions, the treatment of which involves separating two tissue surfaces and their support away from one another. Depending on the condition being treated, the tissue surfaces may be opposed or contiguous and may be bone, skin, soft tissue, or a combination thereof. (U.S. Pat. No. 6,595,998).

Outside of the medical field, there is also often a need to provide a structural element that keeps two surfaces away from each other. In certain environments, the delivery of such structural element into the destination must be through a small opening or an access port.

Therefore, an unfulfilled need still exists for effective, economical, and simple methods of delivery of structural elements to a particular destination through a small opening.

### SUMMARY OF THE INVENTION

Accordingly, it is an object of the present invention to provide simple and effective devices and methods of a radial delivery of a structural element. More particular, it is an object of the invention to provide devices and methods for percutaneous fixation of a spinal compression fracture and reexpanding a partially collapsed vertebral body.

These and other objects are achieved in the present invention by utilizing a device for the delivery of a structural element to a destination. The device comprises a channel and a plunger slidably positioned inside the channel. The channel has a proximal end, a distal end, and a barrier formed across the channel at the distal end. The plunger is adapted to move the structural element axially through the channel from the proximal end to the distal end and to push it radially into the destination.

In one embodiment, the device is used for percutaneous fixation of a spinal compression fracture and reexpanding a partially collapsed vertebral body. In this embodiment, the structural element may be selected from a group consisting of wafers, rod-like structures, plugs, pledgets of bone matrix material, cadaver bone, and a patient's autologous bone.

In one embodiment, the channel has a side wall and a window formed through the side wall adjacent to the distal end. In this embodiment, the plunger is adapted to push the structural element radially through the window into the destination.

In another embodiment, an expandable sack is removably attached to the window in a way such that when the structural elements are pushed radially through the window, they drop into the sack. Thus, in this embodiment, the structural elements are placed into the destination in the sack.

In another aspect, the present invention provides another device for percutaneous fixation of a spinal compression fracture and reexpanding a partially collapsed vertebral body. The device comprises: (i) a channel having a side wall, an open proximal end, and an open distal end; (ii) an expandable sack circumferentially removably attached to the open distal end, wherein an opening in the sack communicates with the channel; and (iii) a plunger slidably positioned inside the channel. The plunger is adapted to move a rod-like structure through the channel into the vertebral body, whereby the rod-like structure is placed into the vertebral body in the sack.

In still another aspect, the present invention provides a method of a radial delivery of a structural element to a destination. The method comprises providing a delivery device described above; loading the structural element into the channel; pushing the structural element axially with the plunger until it reaches the barrier at the distal end; and applying a radial force to the plunger, whereby the plunger pushes the structural element radially into the destination.

The method may be used for percutaneous fixation of a spinal compression fracture and reexpanding a partially collapsed vertebral body in a subject. In one embodiment, rod-like structures are used as a structural element.

In yet another aspect, the present invention provides a kit for percutaneous fixation of a spinal compression fracture and reexpanding a partially collapsed vertebral body in a subject. The kit comprises a plurality of structural elements suitable for insertion into the vertebral body; and a delivery device described above.

The above-described devices and methods of the present invention provide a number of unexpected advantages over the existing delivery devices and methods. The devices have a simple construction and are easy to use. The devices and methods call for structural elements to be deposited within the target “sideways,” thus avoiding antegrade force from being applied to the structural element. This, in turn, avoids inadvertent perforation of the anterior cortex of the target vertebral body by the structural element.

Also, when rod-like structures are being deposited within the target vertebral body, with their gradual and progressive deposition, the height of the partially collapsed vertebral body gradually increases. Such gradual increase makes the procedure safer and easier to perform.

The invention is defined in the appended claims and is described below in its preferred embodiments.

### DESCRIPTION OF THE FIGURES

The above-mentioned and other features of this invention and the manner of obtaining them will become more apparent, and will be best understood by reference to the following description, taken in conjunction with the accompanying drawings, in which:

FIGS. 1a-1c schematically show the operation of the delivery device in accordance with one embodiment of the present invention.

FIGS. 2a and 2b schematically show the operation of the delivery device in accordance with another embodiment of the present invention.

FIGS. 3a-3c schematically show delivery devices in accordance with several embodiments of the present invention.

FIGS. 4a and 4b schematically show the delivery device with an expandable sack in accordance with one embodiment of the present invention. The operation of device (FIG. 4a) and some configurations of the sack (FIG. 4b) are shown.

FIG. 5 illustrates a method of the percutaneous fixation of a compression fracture comprising a step of rotating the delivery device in accordance with one embodiment of the present invention.

FIGS. 6a-6d depict some tools that may be used to form a passage through the pedicles for placement of the delivery device of the present invention (FIGS. 6a-6c) and a self-installing delivery device (FIG. 6d).

### DETAILED DESCRIPTION OF THE INVENTION

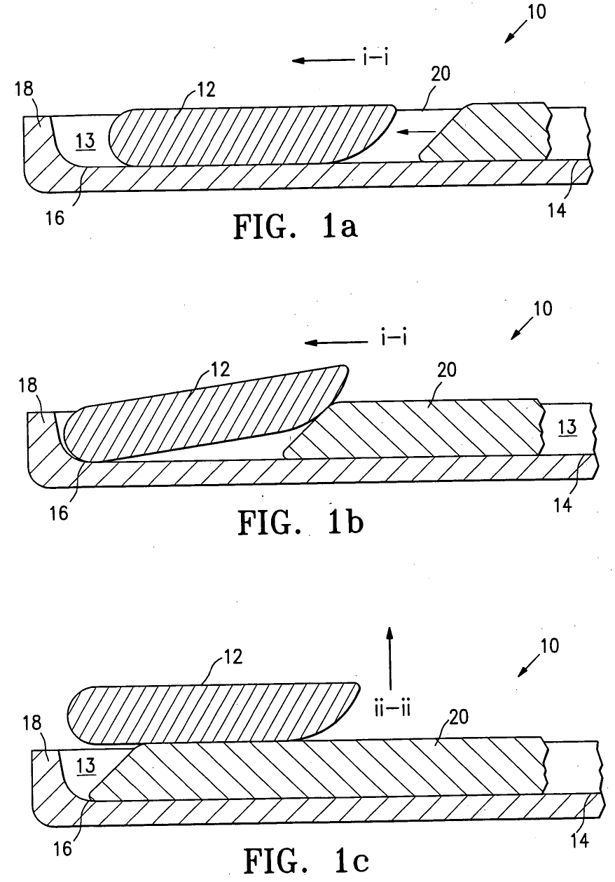

Referring to FIG. 1, in one aspect, the present invention is directed to a device 10 for delivery of a structural element 12 to a destination. The device comprises a channel 13 with a proximal end 14, a distal end 16, and a barrier 18 formed across the channel 13 at the distal end 16. The device further comprises a plunger 20 slidably positioned inside the channel 13. The plunger 20 is adapted to move the structural element 12 axially i-i through the channel from the proximal end 14 to the distal end 16 and to push it radially ii-ii into the destination.

For the purposes of the present invention, the terms “radial” or “radially” mean positioned, occurring, applying force or moving along a ray radiating outward from the longitudinal axis of the channel 13. Although upward radial movement ii-ii is shown in FIG. 1c, it is to be understood that the phrase “push it radially” is not limited to upward movement of the plunger, but includes all possible radial movements according to the definition of the term “radial” given above.

A variety of applications is possible for the delivery device 10 of the present invention. For example, it may be used to deliver a broad range of agents and structural elements to different sites within the body. Within the vertebral body and other bones, the device may be used to deposit wafers, rod-like structures, plugs or pledgets of bone matrix material, cadaver bone or the patient's autologous bone with or without bone morphogenetic protein (BMP). Radiophamaceuticals/radiation sources, chemotherapeutic drugs, or biological agents (such as stem cells or gene therapy vectors) for the treatment of cancer or a host of degenerative diseases may also be deposited within bone, bodily organs or the brain of a human or an animal. These and other structural elements and agents are known and commonly used by those skilled in the medical and veterinary arts. Accordingly, the known features of such structural elements and agents will not be discussed here in detail.

Outside of the field of medicine, this device employing the radial delivery of some agent or a structural element may be used in manufacturing or mining, especially where one needs to fill a cavity through a small opening or an access port.

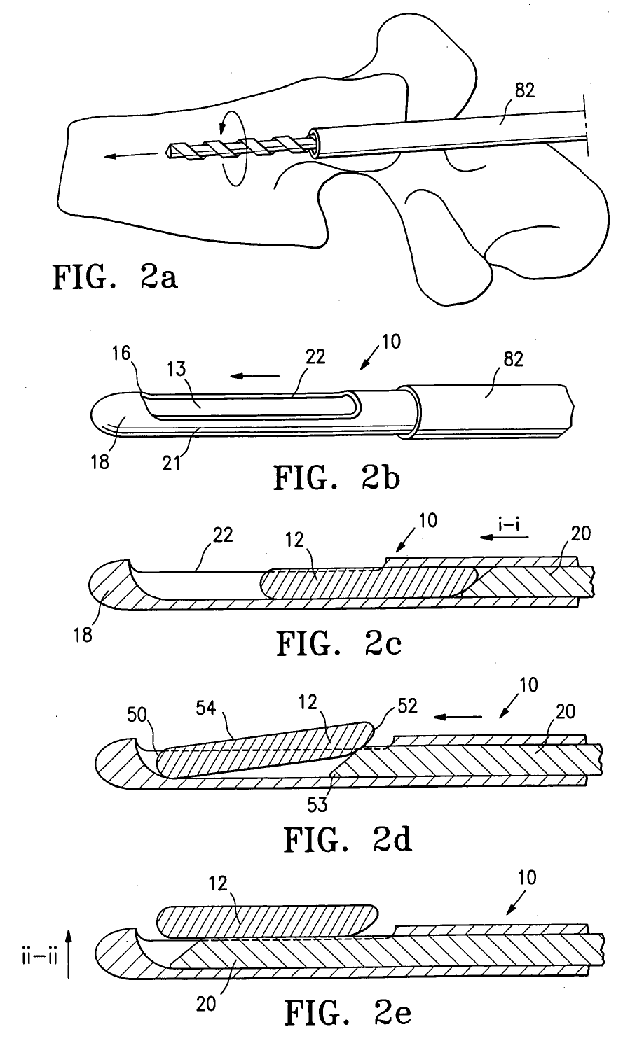

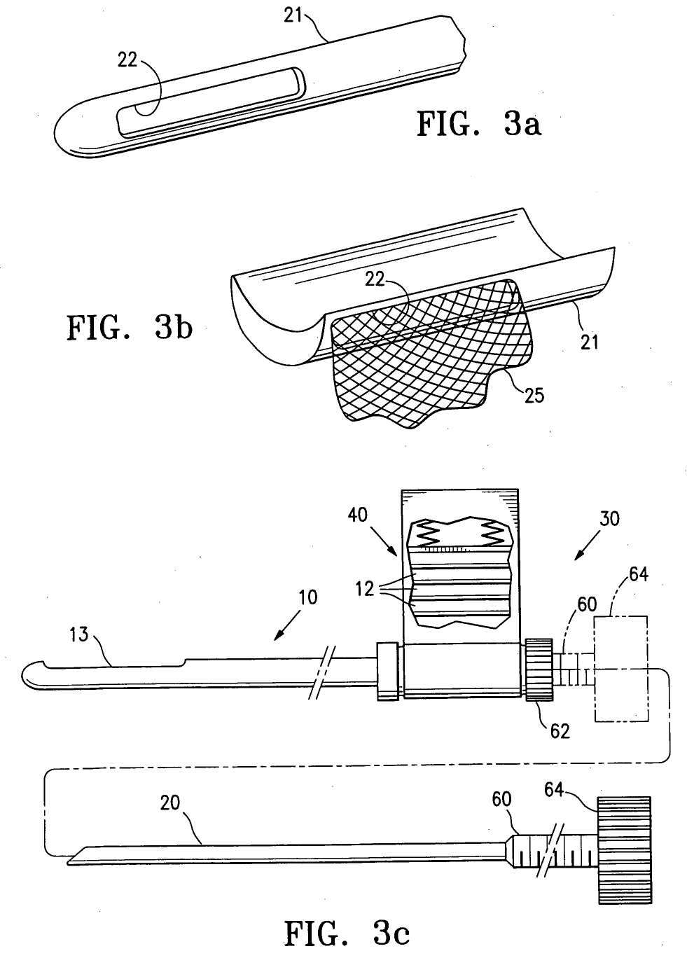

Referring to FIGS. 2b-2e, in one embodiment, the channel 13 has a side wall 21 and a window 22 formed through the side wall adjacent to the distal end 16. The plunger 20 is adapted to push the structural element 12 radially through the window into the destination (FIGS. 2d-2e). The channel may be inside a pipe-like holder (FIG. 3a) or inside a half-pipe holder (FIG. 3b). Preferably, the window 22 has a length that is about the same as a length of the structural element. In one embodiment, the delivery device 10 comprises at least one additional window.

Referring to FIG. 3b, in another embodiment, the delivery device 10 comprises an expandable sack 25 removably attached to the window 22 in a way such that when the structural elements are pushed radially through the window, they drop into the sack. According to this embodiment, the structural elements are placed into the desired destination in the sack 25. The sack may separate from the delivery device when a predetermined number of structural elements is accumulated in the sack or/and the delivery device 10 is withdrawn from the body.

Referring to FIGS. 2b-2e, in one embodiment, the structural element 12 is a rod-like structure. The rod-like structure may be an intramedullary rod made of a metal, an alloy, a polymer, a cadaver bone material, or a composite. In one embodiment shown in FIGS. 2d and 2e, the rod-like structure has a front end 50 and a back end 52. The plunger 20 engages the back end 52 of the rod-like structure 12 with its first end 53 to move the rod-like structure axially through the channel. To facilitate the engagement between the plunger 20 and the rod-like structure 12, the first end 53 of the plunger and the back end of the rod-like structure 52 are beveled. In one embodiment, to improve sliding of the rod-like structure through the channel, the rod-like structure 12 has a rounded front end 50.

The rod-like structure, the channel and the plunge may be made of any durable material including, but not limited to, the same or different metal or plastic materials. The rod-like structures may be of any shape as long as they fit in and may be moved through the channel first axially and, then, radially. For example, the rod-like structures may have a cross-section that is normal to its longitudinal axis and wherein the cross-section is selected from a group consisting of circles, ovals, polygons, and figures combining curved and straight sides. In one embodiment, the rod-like structures have a length of 1.5-2 cm and a diameter of about 13 gauge.

In another embodiment, the rod-like structure has a leading edge 54 on its side to facilitate its entry into the vertebral body. In yet another embodiment, the channel, the rod-like structure, or both have a lubricated coating that facilitates movement of the rod-like structure through the channel 13.

Referring to FIG. 3c, the delivery device 10 may further comprise a plunger-advancing mechanism 30 in communication with the plunger 20. In one embodiment, the plunger 20 is advanced with the aid of a screw-like mechanism, which generates the radial force necessary to extrude the structural element into the desired destination, such as vertebral body. In this embodiment, the plunger has a threaded portion 60 with an external thread and the plunger-advancing mechanism comprises a sleeve 62, defining an interior channel having an internal thread complimentary to the external thread of the plunger. The threaded portion of the plunger 60 is received through the sleeve 62 whereby the external thread of the plunger mates with the internal thread of the sleeve. The plunger advancing mechanism further comprises a nut or a handle 64 attached to the threaded portion of the plunger 60, wherein rotation of the nut or the handle 64 advances the plunger 13 forward into the channel or retrieves it therefrom.

In one embodiment, the screw-like device provides sufficient radial force to plunger to enable its radial movement. In another embodiment, the delivery device comprises an additional mechanism, such as a spring-loaded mechanism, that enables radial movement of the plunger 20 inside the channel 13. In still another embodiment, a portion of the channel 13 containing the window is detachable from the rest of the channel and may be left in the subject.

The delivery device 10 may further comprise a feeder 40 operatively connected with the channel 13. The feeder 40 is adapted for holding a plurality of the structural elements 12, such as rod-like structures, and for placing them into the channel sequentially and on demand. The feeder may further comprise a trigger mechanism for forcing structural elements 12 into the channel 13. Those skilled in the art are familiar with various types of feeders and would be able to select one suitable for the instant application based on the disclosure provided herein. For example, the feeder may work similar to an ammunition clip for an automatic weapon. Each time the plunger is sufficiently withdrawn, it allows another structural element to be spring-loaded into the channel 13. Another suitable feeder is described in the U.S. Pat. No. 6,595,998, the entire content of which is incorporated herein by the reference.

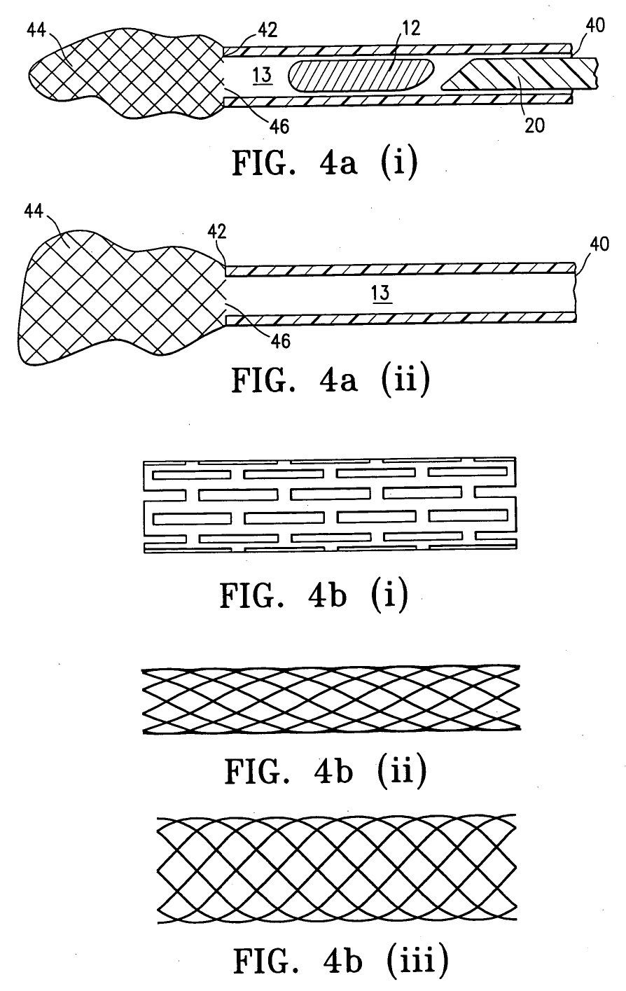

In reference to FIGS. 4a and 4b, in another aspect, the present invention provides a device for percutaneous fixation of a spinal compression fracture and reexpanding a partially collapsed vertebral body in a subject by seating a rod-like structure within the vertebral body. The device comprises a channel 13 having an open proximal end 40 and an open distal end 42. The device also includes an expandable sack 44 circumferentially removably attached to the open distal end 42 in a way that ensures that an opening in the sack 46 communicates with the channel 13. A plunger 20 is slidably positioned inside the channel 13. The plunger is adapted to move a structural element, such a rod-like structure 12, through the channel into the vertebral body, whereby the rod-like structure is placed into the vertebral body in the sack 44.

The sack may be made of any durable and biocompatible material. For example, the material for the sack 44 may be selected from a group consisting of a metal, a metal alloy, or a plastic. Some embodiments of the sack 44 of the present inventions include, but are not limited to, a thin-walled tantalum or elgiloy metal tube with multiple linear fenestrations (FIG. 4b(i)); a compressed tube constructed from a thin braided metal or metal alloy wires (FIG. 4b(ii)); or a polymer mesh (FIG. 4c(iii)). Optionally, the sack may be removably attached to distal end 42 by any suitable method, including, but not limited to, a thin latex, adhesive, or an elastic sheath.

In another aspect, the present invention provides a method of a radial delivery of a structural element to a destination. The method, which is illustrated in FIGS. 2a-2e, comprises: (a) providing a delivery device 10 having a channel 13 with a distal end 16 and a barrier 18 formed across the channel at the distal end; and a plunger 20 slidably positioned inside the channel 13 (FIGS. 2b and 2c); (b) loading the structural element 12 into the channel; (c) pushing the structural element 12 axially (marked as a direction i-i)with the plunger 20 (FIG. 2c) until it reaches the barrier 18 at the distal end (FIG. 2d); and (d) applying a radial force ii-ii to the plunger 20, whereby the plunger 20 pushes the structural element 12, such as a rod-like structure, radially into the destination.

In the embodiment shown in FIGS. 2a-2e, the channel has a side wall 21 and a window 22 formed through the side wall adjacent to the distal end. In this embodiment, the method further comprises steps of sliding the plunger between the structural element and the side wall and pushing the structural element radially through the window 22. The method of the present invention may also include a step of placing the delivery device proximate to the target destination, such as a collapsed vertebral body, and with the window facing the destination. In another embodiment, the method further comprises a step of withdrawing the plunger from the channel after pushing one structural element through the window to allow the loading of the next structural element.

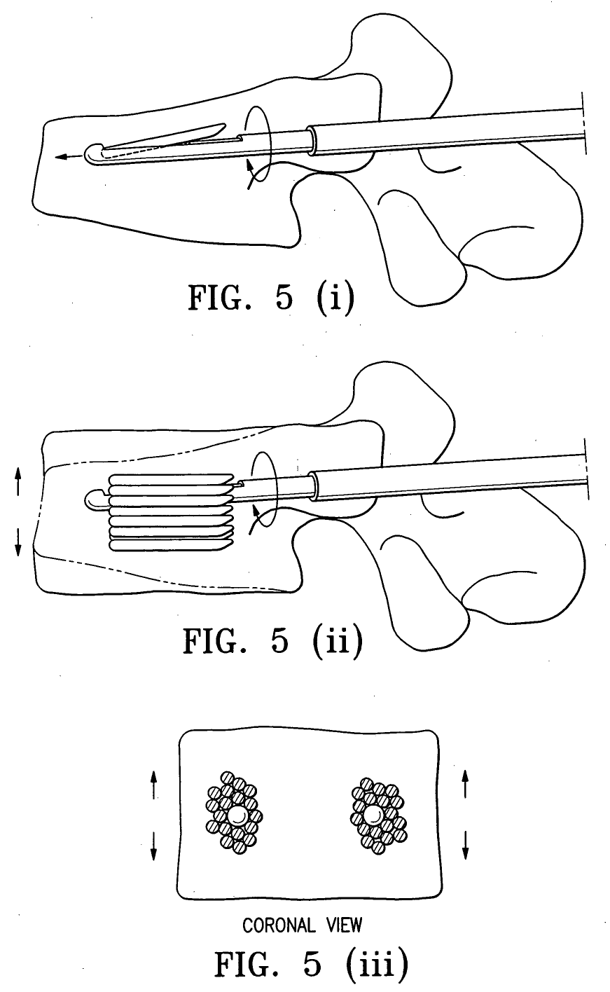

A plurality of the structural elements may be deployed into the destination by sequentially repeating steps (b)-(e) of the method above with each structural element. Referring to FIG. 5, in yet another embodiment, the method further comprises a step of rotating the delivery device (FIG. 5(ii)) after placing one structural element (FIG. 5(i)) and before placing next structural element into a different location within the destination. Such rotation of the delivery device would direct the placement of structural elements, such as minirods, either cephalad or caudad, in order to achieve the desired effect of restoring the vertebral body's height. As shown in FIG. 5(iii), the turning the delivery device after placing a structural element, ensures more even, multi-directional distribution of the structural elements in the target area, such as bone.

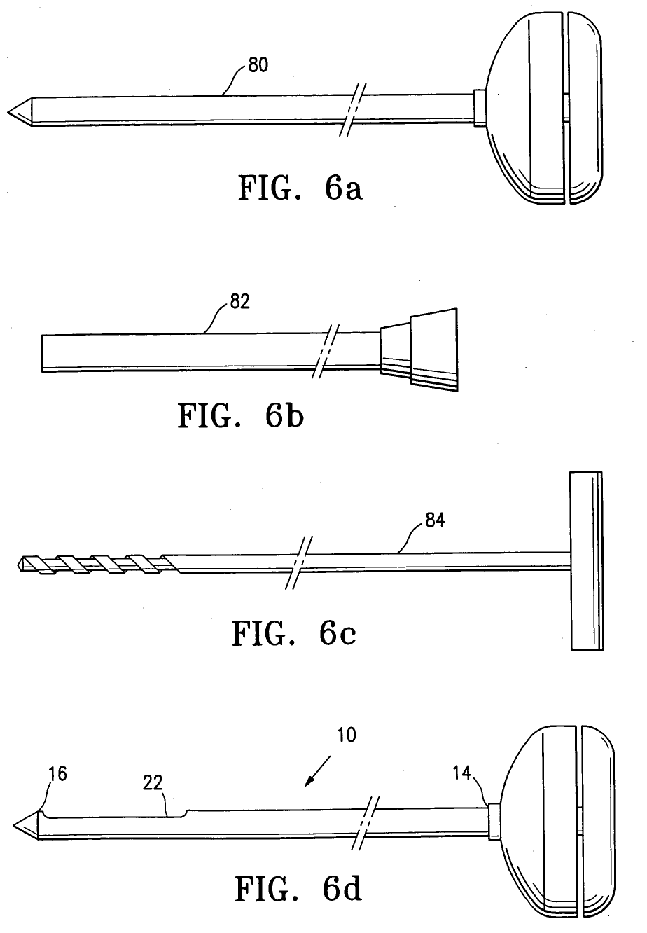

In one embodiment, the delivery device is used for stabilizing spinal compression fractures and reexpanding partially collapsed vertebral bodies. In this embodiment, the device may be inserted bilaterally transpedicularly into the affected vertebral body. This may be accomplished under fluoroscopic guidance by first driving 11-gauge bone biopsy needles 80 (FIG. 6a) through the pedicles and into the affected body. The stylet of these needles is replaced with an 0.038″ guidewire or surgical K-wire over which is inserted an 8-gauge metal sheath 82 with a central metal dilator (FIGS. 2a and 6b). The sheath is inserted to the pedicle-body junction bilaterally. A small hand drill 84 may be inserted through these sheaths and rotated to create a pathway anterior to the metal sheaths for the subsequent insertion of an 11-gauge metal delivery tube device to deliver and deploy multiple rod-like structures.

In another embodiment, the present invention provides a self-installing delivery device 10 having a sharp-needle like distal end 16 that facilitates its insertion without a need for all or some of the installation tools described above. The delivery device also has window 22 for radial ejection of structural elements from the delivery device.

The structural elements may be “minirods” having a length of 1.5-2 cm and a diameter of about 13 gauge. In one embodiment, the delivery device for the minirod is an 11-gauge metal tube with a sealed distal end and a distal side window with a length approximately the same as the minirod. Minirods may be sequentially deployed within the vertebral body by pushing the minirod to the distal end of the delivery tube with a plunger advanced forward by a screw mechanism, much the same as the screw mechanism used in balloon angioplasty inflation syringes. Once the minirod reaches the side window zone of the distal delivery tube, further advancement of the plunger causes the minirod to be pushed out radially from the lumen of the delivery tube, compacting the soft demineralized bone of the partially collapsed vertebral body. Accordingly, advancing the plunger completely to the end of the lumen of the delivery tube results in the total extrusion of the minirod from the delivery tube. The plunger may then be withdrawn from the delivery lumen (by disengaging the screw mechanism and pulling it back), thus allowing the insertion of the next minirod.

At the conclusion of the procedure, the 8-gauge metal sheaths are removed from the pedicles and the skin incisions are sterilely dressed. The entire procedure may be performed under fluoroscopic guidance with the patient in the prone position. I.V. sedation with local anesthesia or general endotracheal anesthesia may be utilized.

In addition to restoring vertebral body height, the structural elements of the present invention may act as multiple intramedullary rods, stabilizing the fractured vertebral body, and increasing its tensile strength.

In another embodiment, the methods of the present invention may further comprise a step of injecting a polymer or a bone matrix material within and around the rod-like structures placed into the vertebral body. The bone matrix material may comprise an osteoconductive or an osteoinductive material, such as bone morphogenetic protein (or BMP). The polymer may be polymethyl methacrylate (PMMA) or a biocompatible polyurethane preparation. Polymers and bone matrix material would act to enhance fracture stabilization and bone tensile strength. The injection may be made through the delivery device prior to its removal.

In another aspect, the present invention provides a kit for percutaneous fixation of a spinal compression fracture and reexpanding a partially collapsed vertebral body in a subject. The kit comprises a plurality of structural elements suitable for insertion into the vertebral body; and a delivery device of the present invention as described above. The structural elements may have the same, or a different, size. Referring, for example, to FIG. 3a, in one embodiment, the structural elements of at least two different sizes are used and the window 22 is sized to accommodate the longest structural element.

It will be apparent to those skilled in the art that various modifications and variations can be made in system and methods of the present invention without departing from the spirit or scope of the inventions. Thus, it is intended that the present invention cover modifications and variations of this invention that come within the scope of the appended claims and their equivalents.

1. A device for delivery of a structural element to a destination, the device comprising:
a channel with a proximal end, a distal end, and a barrier formed across the channel at the distal end;
a plunger slidably positioned inside the channel, wherein the plunger is adapted to move the structural element axially through the channel from the proximal end to the distal end and to push it radially into the destination.

2. The device of claim 1, wherein the structural element is selected from a group consisting of wafers, rod-like structures, plugs, pledgets of bone matrix material, cadaver bone, and a patient's autologous bone.

3. The device of claim 2, wherein the structural element further comprises bone morphogenetic protein (BMP).

4. The device of claim 1, wherein the destination is an organ, brain, or bone of a human or an animal.

5. The device of claim 1, wherein the structural element further comprises radiophamaceuticals, radiation sources, chemotherapeutic drugs, biological agents or their combinations.

6. The device of claim 1, wherein the channel has a side wall and a window formed through the side wall adjacent to the distal end and wherein the plunger is adapted to push the structural element radially through the window into the destination.

7. The device of claim 6, wherein the channel is formed inside a pipe or a half-pipe holder.

8. The device of claim 6 comprising at least one additional window.

9. The device of claim 6 further comprising an expandable sack removably attached to the window in a way such that when the structural elements are pushed radially through the window, they drop into the sack, whereby the structural elements are placed into the destination in the sack.

10. The device of claim 1 further comprising a plunger advancing mechanism in communication with the plunger.

11. The device of claim 1 further comprising a mechanism that enables radial movement of the plunger inside the channel.

12. The device of claim 1, wherein the distal end is in a form of a needle.

13. The device of claim 1 further comprising a feeder operatively connected with the channel, wherein the feeder is adapted for holding a plurality of the structural elements and for placing them into the channel sequentially and on a demand.

14. A device for percutaneous fixation of a spinal compression fracture and reexpanding a partially collapsed vertebral body in a subject by seating a rod-like structure within the vertebral body, the device comprising:
a channel having an open proximal end and an open distal end;
an expandable sack circumferentially removably attached to the open distal end, wherein an opening in the sack communicates with the channel; and
a plunger slidably positioned inside the channel, wherein the plunger is adapted to move the rod-like structure through the channel into the vertebral body, whereby the rod-like structure is placed into the vertebral body in the sack.

15. The device of claim 14, wherein the sack is made of a metal, a metal alloy, or a plastic.

16. The device of claim 15, wherein the sack comprises a thin-walled tantalum or elgiloy metal tube with multiple linear fenestrations; a compressed tube constructed from a thin braided metal or metal alloy wires; or a polymer mesh.

17. The device of claim 14, wherein the sack is attached to the distal end by a thin latex, adhesive, or elastic sheath.

18. A device for percutaneous fixation of a spinal compression fracture and reexpanding a partially collapsed vertebral body in a subject by seating a rod-like structure within the vertebral body, the device comprising:
a channel with a proximal end, a distal end, and a barrier formed across the channel at the distal end; and
a plunger slidably positioned inside the channel, wherein the plunger is adapted to move the structural element axially through the channel from the proximal end to the distal end and to push it radially into the vertebral body.

19. The device of claim 18, wherein the rod-like structure is an intramedullary rod made of a metal, an alloy, a polymer, a cadaver bone material, or a composite.

20. The device of claim 18, wherein the rod-like structure has a front end and a back end and wherein the plunger engages the back end of the rod-like structure with a first end to move the rod-like structure axially through the channel and wherein the first end of the plunger and the back end of the rod-like structure are beveled.

21. The device of claim 20, wherein the rod-like structure has a rounded front end.

22. The device of claim 18, wherein the channel has a side wall and a window formed through the side wall adjacent to the distal end and wherein the plunger is adapted to push the rod-like structure radially through the window into the destination.

23. The device of claim 22, wherein the window has a length that is about the same as a length of the rod-like structure.

24. The device of claim 22, wherein the channel is formed inside a pipe or a half-pipe holder.

25. The device of claim 22 further comprising an expandable sack removably attached to the window in a way such that when the rod-like structures are pushed radially through the window, they drop into the sack, whereby the rod-like structures are placed into the vertebral body with the sack.

26. The device of claim 25, wherein the sack is made of a metal, a metal alloy, or a plastic.

27. The device of claim 25, wherein the sack comprises a thin-walled tantalum or elgiloy metal tube with multiple linear fenestrations; a compressed tube constructed from a thin braided metal or metal alloy wires; or a polymer mesh.

28. The device of claim 25, wherein the sack is attached to the window by a thin latex, adhesive, or elastic sheath.

29. The device of claim 18, wherein the rod-like structure, the channel and the plunge are made of the same or different metal or plastic materials.

30. The device of claim 29, wherein the rod-like structure has a cross-section that is normal to its longitudinal axis and wherein the cross-section is selected from a group consisting of circles, ovals, polygons, and figures combining curved and straight sides.

31. The device of claim 18, wherein the distal end is shaped as a needle.

32. The device of claim 18, wherein the rod-like structure has a leading edge on its side to facilitate its entry into the vertebral body.

33. The device of claim 18, wherein the channel, the rod-like structure, or both have a lubricated coating that facilitates movement of the rod-like structure through the channel.

34. The device of claim 18 further comprising a plunger advancing mechanism in communication with a first end of the plunger.

35. The device of claim 34, wherein the plunger has a threaded portion adjacent to the first end, the threaded portion having an external thread and wherein the plunger advancing mechanism comprises:
a sleeve defining an interior channel having an internal thread complimentary to the external thread of the plunger, wherein the threaded portion of the plunger is received through the sleeve whereby the external thread of the plunger mates with the internal thread of the sleeve; and
a nut or a handle attached to the threaded portion of the plunger, wherein rotation of the nut or the handle advances the plunger forward into the channel or retrieves it therefrom.

36. The device of claim 18 further comprising a mechanism that enables radial movement of the plunger inside the channel.

37. The device of claim 36, wherein the mechanism comprises a spring.

38. The device of claim 18 further comprising a feeder operatively connected with the channel, wherein the feeder is adapted for holding a plurality of the rod-like structures and for placing them into the channel sequentially and on a demand.

39. The device of claim 38, wherein the feeder further comprises a trigger mechanism for forcing the rod-like structures into the channel.

40. The device of claim 18, wherein a portion of the channel is detachable from the rest of the channel and may be left in the subject.

41. A method of a radial delivery of a structural element to a destination, the method comprising:
(a) providing a delivery device having
a channel with a proximal end, a distal end, and a barrier formed across the channel at the distal end; and
a plunger slidably positioned inside the channel;
(b) loading the structural element into the channel;
(c) pushing the structural element axially with the plunger until it reaches the barrier at the distal end; and
(d) applying a radial force to the plunger, whereby the plunger pushes the structural element radially into the destination.

42. The method of claim 41, wherein the channel has a side wall and a window formed through the side wall adjacent to the distal end and wherein the method further comprises steps of sliding the plunger between the structural element and the side wall and pushing the structural element radially through the window.

43. The method of claim 42, further comprising a step of placing the delivery device proximate to the destination and with the window facing the destination.

44. The method of claim 41, wherein a plurality of the structural elements is deployed into the destination by sequentially repeating steps (b)-(e) with each structural element.

45. The method of claim 44 further comprising a step of withdrawing the plunger from the channel after pushing one structural element through the window to allow the loading of the next structural element.

46. The method of claim 41, further comprising a step of rotating the delivery device after placing one structural element and before placing next structural element into a different location within the destination.

47. A method of percutaneous fixation of a spinal compression fracture and reexpanding a partially collapsed vertebral body in a subject, the method comprising:
(a) providing a delivery device having
a channel with a distal end and a barrier formed across the channel at the distal end; and
a plunger slidably positioned inside the channel;
(b) loading a rod-like structure into the channel;
(c) pushing the rod-like structure with the plunger until it reaches the barrier at the distal end; and
(d) applying a radial force to the plunger, whereby the plunger pushes the rod-like structure through the window into the partially collapsed vertebral body.

48. The method of claim 47, wherein the channel has a side wall and a window formed through the side wall adjacent to the distal end, wherein the method further comprises steps of sliding the plunger between the rod-like structure and the side wall of the holder and pushing the rod-like structure through the window.

49. The method of claim 48, further comprising a step of placing the delivery device proximate to the collapsed vertebral body and with the window facing a desired location for placement of the rod-like structure.

50. The method of claim 46, wherein step (d) comprises placing the rod-like structures bilaterally transpedicularly

51. The method of claim 50 further comprising a step of providing a pathway through the pedicle into an affected body for placement of the delivery device.

52. The method of claim 51 wherein the pathway is formed by:
(a) driving a bone biopsy needle through the pedicle and into the affected body;
(b) replacing the stylet of the needle with a guidewire or a surgical K-wire;
(c) bilaterally inserting a sheath with a central dilator over the guidewire or a surgical K-wire to the pedicle-body junction; and
(d) drilling the pathway anterior to the metal sheaths.

53. The method of claim 47, wherein a plurality of the rod-like structures is deployed into the vertebral body by sequentially repeating steps (b)-(d) with each rod-like structure.

54. The method of claim 53 further comprising a step of withdrawing the plunger from the channel after pushing one rod-like structure through the window to allow the loading of the next rod-like structure.

55. The method of claim 47, further comprising a step of rotating the delivery device after placing one rod-like structure to place next the rod-like structure into a different location in the vertebral body.

56. The method of claim 47, further comprising a step of injecting a polymer or a bone matrix material within and around the rod-like structures placed into the vertebral body.

57. The method of claim 56, wherein the bone matrix material comprises an osteoconductive or an osteoinductive material.

58. The method of claim 57, wherein the osteoinductive material comprises BMP.

59. The method of claim 57, wherein the polymer is polymethyl methacrylate (PMMA) or polyurethane.

60. A kit for percutaneous fixation of a spinal compression fracture and reexpanding a partially collapsed vertebral body in a subject, comprising:
a plurality of structural elements suitable for insertion into the vertebral body; and
a delivery device having a channel with a proximal end, a distal end, and a barrier formed across the channel at the distal end; and a plunger slidably positioned inside the channel, wherein the plunger is adapted to move the structural element axially through the channel from the proximal end to the distal end and to push it radially into the vertebral body.

61. The kit of claim 60, wherein the channel has a side wall and a window formed through the side wall adjacent to the distal end, wherein the plunger is adapted to push the structural element radially through the window into the vertebral body.

62. The kit of claim 60, wherein the structural element is selected from a group consisting of wafers, rod-like structures, plugs, pledgets of bone matrix material, cadaver bone, and a patient's autologous bone.

63. The kit of claim 80, wherein the structural elements have the same size.

64. The kit of claim 80, wherein the structural elements have at least two different sizes and the window is sized to accommodate the longest structural element.
